(19) United States
(12) Patent Application Publication
(10) Pub. No.: US 2006/0159746 A1
(43) Pub. Date: Jul. 20, 2006

(54) Compositions comprising fatty acids and amino acids

(76) Inventors: John P. Troup, Plymouth, MN (US); Michael Beer, Koniz (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/546,942

(22) PCT Filed: Feb. 23, 2004
(86) PCT No.: PCT/EP04/01763

§ 371 Date: Aug. 25, 2005

Related U.S. Application Data
(60) Provisional application No. 60/455,453, filed on Mar. 18, 2003.

(51) Int. Cl. A61K 31/715; A61K 9/20; A61K 31/202; A61K 31/198

(52) U.S. Cl. 424/464; 514/54; 514/561; 514/565; 514/567; 514/560; 514/563

(57) ABSTRACT

The invention relates to a combination, such as a combined preparation or pharmaceutical or nutritional composition, respectively, which comprises at least one cis-polyunsaturated fatty acid, at least one amino acid, and optionally at least one diabetes medicine for simultaneous, separate or sequential use in the prevention, delay of progression or treatment of diseases, especially metabolic disorders and in particular type 2 diabetes and diseases and conditions associated with diabetes.

The present invention concerns a combination, such as a combined preparation or pharmaceutical or nutritional composition, which comprises at least one cis-polyunsaturated fatty acid and at least one amino acid in free form or pharmaceutically acceptable salt form, optionally a soluble fiber and a non-glucose carbohydrate, and optionally at least one diabetes medicine, for simultaneous, separate or sequential use in the prevention, delay of progression or treatment of metabolic disorders and in particular type 2 diabetes and disease and conditions associated with diabetes; the use of such combination for the preparation of a medicament for the prevention, delay of progression or treatment of metabolic disorders; the use of such combination for the cosmetic treatment of a mammal in order to effect a cosmetically beneficial loss of body weight; a method of prevention, delay of progression or treatment of metabolic disorders and in particular type 2 diabetes and disease and conditions associated with diabetes in warm-blooded animals; a method of improving bodily appearance of a warm-blooded animal; to a pharmaceutical or nutritional composition which comprises such combination and a pharmaceutically or nutritionally acceptable carrier; and to a process of making such pharmaceutical or nutritional composition.

The goals of therapy for type 1 and type 2 diabetes are to eliminate symptoms related to hyperglycemia, reduce or eliminate the long-term microvascular or macrovascular complications of diabetes, and allow the patient to achieve as normal life-style as possible. Type 2 diabetes is characterized by both increased peripheral insulin resistance and abnormal insulin secretion. At least two abnormalities of insulin secretion are recognized: in the first phase insulin is both delayed and inadequate in the face of elevated circulating glucose levels and in the second phase insulin secretion is lost. Several metabolic, hormonal, and pharmacological entities are known to stimulate insulin secretion including glucose, amino-acids and gastrointestinal peptides.

Type 2 diabetes is a prevalent disease associated with increased life expectancy, increase in obesity and decrease of physical activity. Clinical strategies are desperately needed to control plasma glucose levels and prevent complications of the disease. In type 2 diabetes, as compared to type 1 diabetes, there is an increased prevalence of cardiovascular risk factors, such as hypertension, dsylipidemia and obesity. A majority of individuals with type 2 diabetes are obese. Clinical studies are ongoing aiming at determining whether or not lifestyle changes or therapeutic intervention at the stage of impaired glucose tolerance (IGT) can prevent the onset of diabetes or whether or not tight metabolic control and the class of therapeutic agents can reduce macrovascular disease in type 2 diabetes. The number of individuals with IGT worldwide is enormous and about 5% of individuals with IGT develop diabetes each year. Any option that can prevent the transformation of IGT into diabetes is highly sought after.

Surprisingly, it has now been found that the effect of a combination of at least one cis-polyunsaturated fatty acid and at least one amino acid in free form or pharmaceutically acceptable salt form, optionally a soluble fiber and a non-glucose carbohydrate, and optionally at least one diabetes medicine, is greater than the effect that can be achieved with either type of combination partner alone, i.e. greater than the effect of a nutritional therapy using only one of the combination partners and as defined herein.

Hence, in one aspect, the present invention pertains to a combination, such as a combined preparation or pharmaceutical or nutritional composition, which comprises

(a) at least one cis-polyunsaturated fatty acid, e.g. at least one of linolenic, linoleic, conjugated linoleic acid, arachidonic, eicosapentaenoic acid or docosahexaenoic acid, and

(b) at least one amino acid in free and/or pharmaceutically or nutritionally acceptable salt form, e.g. at least one of phenylalanine, valine, arginine, leucine or isoleucine, optionally

(c) a soluble fiber and a non-glucose carbohydrate, and optionally

(d) at least one diabetes medicine,

in which the cis-polyunsaturated fatty acid may be present in free form or in form of an oil or fat and the amino acid in free form or pharmaceutically or nutritionally acceptable salt form may be present alone or in combination with intact protein form, optionally further comprising one or more pharmaceutically or nutrionally acceptable carrier, for simultaneous, separate or sequential use in the prevention, delay of progression or treatment of metabolic disorders and in particular type 2 diabetes and disease and conditions associated with diabetes. Such a combination is preferably a combined preparation or a pharmaceutical or nutritional composition.

A combination which comprises a cis-polyunsaturated fatty acid and an amino acid, optionally a soluble fiber and a non-glucose carbohydrate, and optionally at least one diabetes medicine, in which the cis-polyunsaturated fatty acid is present in free form or in form of an oil or fat and the amino acid is present in free form or in form of a pharmaceutically or nutritionally acceptable salt alone or in combination with intact protein, and optionally one or more pharmaceutically or nutritionally acceptable carrier, is referred to hereinafter as a combination of the invention.

The term “combined preparation”, as used herein defines especially a “kit of parts” in the sense that the combination partners as defined above can be administered independently, i.e. separately, or by use of different fixed combinations, e.g. with distinguished amounts of the combination partners. A therapeutically effective amount of each of the combination partners or a jointly effective amount, preferably synergistically effective amount of the combination partners may be administered simultaneously or sequentially at different time points and in any order. The ratio of the total amounts of the combination partners to each other to be administered in the combined preparation can vary, e.g. in order to cope with the needs of a patient sub-population to be treated or the needs of the single patient which different needs can be due to age, sex, body weight, etc. of the patients. Preferably, there is at least one beneficial effect, e.g., a mutual enhancing of the effect of the combination partners, in particular a synergism, e.g. a more than additive effect. Even more preferably there are additional advantageous effects, such as less side effects, potentiation, i.e. a combined therapeutical effect in a non-effective dosage of one or more of the combination partners, especially a strong synergism of the combination partners.

In one aspect the present invention provides a pharmaceutical or nutritional composition comprising a quantity, which is jointly effective in prevention, delay of progression or treatment of metabolic disorders and in particular type 2 diabetes and disease and conditions associated with diabetes, of the combination of the invention. In this composition, the combination partners as defined above can be administered together, one after the other or separately in one combined unit dosage form or in two separate unit dosage forms. The unit dosage form may also be a fixed combination.

In particular, the present invention relates to a method of treating metabolic disorders, more especially diabetes and in particular type 2 diabetes mellitus, or a disease or condition associated with diabetes comprising administering to a warm-blooded animal in need thereof a jointly therapeutically effective amount of a combined preparation comprising a cis-polyunsaturated fatty acid and an amino acid, e.g. at least one of phenylalanine, valine, arginine, leucine or isoleucine, optionally a soluble fiber and a non-glucose carbohydrate, and optionally a diabetes medicine, in which the cis-polyunsaturated fatty acid is present in free form or in form of an oil or fat and the amino acid is present in free form or in form of a pharmaceutically or nutritionally acceptable salt alone or in combination with intact protein.

As used herein the term metabolic disorders encompasses diabetes, type 2 diabetes and diseases or conditions associated with diabetes mellitus.

“Diseases and conditions associated with diabetes mellitus” as defined in this application comprise, but are not restricted to hyperglycemia, hyperinsulinaemia, hyperlipidaemia, insulin resistance, impaired glucose metabolism, obesity, diabetic retinopathy, macular degeneration, cataracts, diabetic nephropathy, glomerulosclerosis, diabetic neuropathy, erectile dysfunction, premenstrual syndrome, vascular restenosis and/or ulcerative colitis. Furthermore, “diseases and conditions associated with diabetes mellitus” comprise, but are not restricted to: coronary heart disease, hypertension, angina pectoris, myocardial infarction, stroke, skin and/or connective tissue disorders, foot ulcerations, metabolic acidosis, arthritis, osteoporosis and in particular conditions of impaired glucose tolerance.

The term “prevention” means prophylactic administration of the combination, such as a combined preparation or pharmaceutical or nutritional composition, to healthy patients to prevent the onset of the diseases and conditions mentioned herein. Moreover, the term “prevention” means prophylactic administration of such combination to patients being in a pre-stage of the disease, especially diabetes, to be treated. The term “delay of progression” used herein means administration of the combination, such as a combined preparation or pharmaceutical or nutritional composition, to patients being in a pre-stage of the disease, especially diabetes, to be treated in which patients a pre-form of the corresponding disease is diagnosed. The term “method of treating” used herein includes a method of prevention of a disease, i.e. the prophylactic administration of the combination, such as a combined preparation or pharmaceutical or nutritional composition, to healthy patients to prevent the onset of the diseases and conditions mentioned herein.

As used herein the meaning of the terms “active agent”, “active ingredient”, “active compound” or in some cases “compound” is to be understood as equivalent.

As used herein, the term cis-polyunsaturated fatty acid (cis-PUFA) refers to a family of carboxylic acids comprising n-3 fatty acids such as alpha-linolenic acid (18:3) (LNA), stearidonic acid, eicosapentaenoic acid (EPA) (20:5), docosapentaenoic acid (22:5) and docosahexaenoic acid (DHA) (22:6), and n-6 fatty acids such as linoleic acid (18:2) (LA), gamma-linolenic acid (18:3), arachidonic acid (20:4), conjugated linoleic acid (CLA), either in free form or in form of an oil or fat. Such cis-polyunsaturated fatty acids are commonly known and readily commercially available. They are present for example in vegetable oils, e.g. canola oil, or fish oils, e.g. concentrated fish oils.

Preferably a combination of eicosapentaenoic acid and docosahexaenoic acid may be used. More preferably canola oil is used.

As used herein, the term amino acid refers to amino acids in free form or pharmaceutically or nutritionally acceptable salt form, e.g. essential amino acids chosen from at least one of isoleucine, leucine, lysine, methionine, phenylalanine, threonine, tryptophane, valine, or histidine, or e.g. conditionally essential amino acids in free form or pharmaceutically or nutritionally acceptable salt form chosen from at least one of tyrosine, cysteine, arginine, or glutamine, alone or in combination with intact protein. Preferably the amino acid may be chosen from at least one of phenylalanine, valine, arginine, leucine and isoleucine. Such amino acids are commonly known and readily commercially available. When intact protein is used in combination with an amino acid in free form or salt form, such intact protein may be chosen from at least one of casein, whey protein, soy protein, collagen or wheat protein.

In one preferred embodiment of the invention the combination of the invention comprises EPA and/or DHA and an amino acid chosen from at least one of phenylalanine, valine, arginine, leucine and isoleucine, most preferably a combination of arginine, leucin and phenylalanine may be used. In another embodiment of the invention, the combination of the invention comprises EPA and/or DHA, and arginine, optionally with intact protein. In a further embodiment of the invention, the combination of the invention comprises oil such as canola oil or fish oil, and arginine, optionally with intact protein.

As used herein the term soluble fiber refers to soluble fibers such as agar, alginates, carubin, pectin, e.g. pectins from fruits and vegetables, e.g. from citrus fruits and apples, and its derivatives, beta-glucan, such as oat beta-glucan, carrageenans, in particular kappa, lambda and iota carrageenans, furcellaran, inulin, arabinogalactan, cellulose and its derivatives, scleroglucan, psyllium, such as psyllium seed husk, mucilages and gums, e.g. commonly available vegetable gums and more particularly konjac gum, xanthan gum, guar gum (guaran gum), locust bean gum, tara bean gum, gum tragacanth, arabic gum, karaya gum, gum ghatti, gellan gum and other related sterculia gum, alfalfa, clover, fenugreek, tamarind flour. Native and modified, e.g. hydrolyzed, soluble fibers may be used. According to the invention, preferably guar gum, e.g. partially hydrolyzed guar gum, e.g. Benefiber®, from Novartis Nutrition Corporation. Such soluble fiber may be used in combination with a non-glucose carbohydrate, e.g. chosen from at least one of galactose, xylose, fructose or mannose, preferably galactose and/or fructose.

As used herein, the term diabetes medicine refers to medicines such as sulfonylureas, biguanides, e.g. metformin, alpha-glucosidase inhibitors, thiazolidinediones, meglitinides, e.g. naglitinide, dipeptidyl peptidase IV (DPP IV) inhibitors, a 4-hydroxyisoleucine (4-HI) source, or D-phenylalanine. When present in the combination, e.g. combined preparation, e.g. nutritional or pharmaceutical composition of the invention, the diabetes medicine preferably may be nateglinide and/or metformin and/or 4-HI. Preferably a combination of cis-polyunsaturated fatty acid and amino acid in free form or pharmaceutically acceptable salt form may be used in co-therapy with a composition comprising nateglinide and/or 4-HI.

In one aspect of the invention there is provided a combination of an amino acid in free form or pharmaceutically acceptable salt form and 4-HI and the use of such a combination for the preparation of a medicament for the prevention, delay of progression or treatment of metabolic disorders or the use of such combination for the cosmetic treatment of a mammal in order to effect a cosmetically beneficial loss of body weight.

In a further aspect of the invention there is provided a pharmaceutical or nutritional composition comprising a quantity, which is jointly therapeutically effective against metabolic disorders, of a combination comprising

(a) at least one of linolenic, linoleic, conjugated linoleic acid, arachidonic, eicosapentaenoic acid or docosahexaenoic acid,

(b) at least one of phenylalanine, valine, arginine, leucine or isoleucine in free and/or salt form, and optionally

(c) at least one diabetes medicine chosen from at least one of nateglinide, metformin or a 4-hydroxy-isoleucine source, and optionally

(d) at least one soluble fibers(s) and/or at lest one non-glucose carbohydrate, and at least one pharmaceutically or nutritionally acceptable carrier.

In yet a further aspect of the invention there is provided a pharmaceutical or nutritional composition comprising

(a) at least one of linolenic, linoleic, conjugated linoleic acid, arachidonic, eicosapentaenoic acid or docosahexaenoic acid, in particular in form of fish oil, canola oil and/or sunflower oil, and

(b) at least one of phenylalanine, valine, arginine, leucine or isoleucine in free and/or salt form, preferably at least one of phenylalanine, arginine or leucine, in free and/or salt form.

The invention further pertains to the use of a such a combination for the cosmetic treatment of a mammal in order to effect a cosmetically beneficial loss of body weight, and/or for use in the prevention, delay of progression or treatment of metabolic disorders, more especially diabetes or a disease or condition associated with diabetes.

Surprisingly, it has been found that the effect of a nutritional composition comprising high % energy as fat and high % energy as protein against metabolic disorders, e.g. obesity, is greater than the effect that can be achieved with a nutritional composition comprising high % energy as carbohydrate.

Hence, in a further aspect the present invention provides a composition comprising high, e.g. about 5% en to about 70% en, e.g. about 10% en to about 60% en or about 40% en to about 70% en, e.g. about 15% en, or about 20% en, or about 50% en or about 60% en or about 65% en, as fat and high, e.g. about 10% en to about 70% en, e.g. about 20% en to about 60% en, or e.g. about 20% en to about 40% en, e.g. about 30% en or about 35% en or about 55% en as amino-nitrogen source, optionally in combination with a diabetes medicine, and the use of such a composition for the preparation of a nutritional composition for the prevention, delay of progression or treatment of metabolic disorders or the use of such combination for the cosmetic treatment of a mammal in order to effect a cosmetically beneficial loss of body weight.

As used herein, the term fat refers to cis-polyunsaturated fatty acid(s) in free form and/or in form of an oil or fat, e.g. in mono-, di-, or tri-glyceride form, e.g. in form of a vegetable oil, e.g. oleic rich oil, such as canola oil, sunflower oil or oleic rich sunflower oil, or fish oil or concentrated fish oil, e.g. fish oil containing about 70% EPA and about 30% DHA.

As used herein, the terms protein or amino-nitrogen source refer to amino acids, in free form or pharmaceutically or nutritionally acceptable salt form, e.g. essential amino acids, e.g. isoleucine, leucine, lysine, methionine, phenylalanine, threonine, tryptophane, valine, or histidine, conditionally essential amino acids, e.g. tyrosine, cysteine, arginine, or glutamine, or non-essential amino acids, e.g. glycine, alanine, proline, serine, glutamic acid, aspartic acid, asparagines, taurine or carnitine, either alone or in combination with intact protein, e.g. casein, whey protein, soy protein, collagen or wheat protein.

As used herein the meaning of the terms “protein” or “amino-nitrogen source” is to be understood as equivalent.

Nateglinide is generically and specifically disclosed in EP 196222, EP 526171, U.S. Pat. No. 5,463,116 and U.S. Pat. No. 5,488,150, in particular in the compound claims and the final products of the working examples, the subject-matter of the final products, the pharmaceutical preparations and the claims is hereby incorporated into the present application by reference to these publications. Comprised are likewise the corresponding stereoisomers as well as the corresponding crystal modifications, e.g. solvates and polymorphs, which are disclosed therein. The term nateglinide as used herein comprises crystal modifications (polymorphs) such as those disclosed in EP 0526171 B1 or U.S. Pat. No. 5,488,510, respectively, the subject matter of which is incorporated by reference to this application, especially the subject matter of claims 8 to 10 as well as the corresponding references to the B-type crystal modification. Preferably, in the present invention the B- or H-type, more preferably the H-type, is used.

The term 4-HI source as used herein refers to at least one of fenugreek, e.g. Trigonella foenum graecum L., seeds; fenugreek extract, e.g. an ethanolic fenugreek extract, e.g. an extract as known and commercially available under the trade name FenuPure® from Adumin, or Limitrol® from Nutricept; a concentrated Fenugreek extract; or 4-HI itself; e.g. as described in EP0587476, U.S. Pat. No. 5,470,879, WO01/15689, WO01/72688, US 20010048952, the contents of which are hereby incorporated into the present application by reference to these publications. Preferably, a fenugreek extract, e.g. an extract containing between about 30% to about 90%, e.g. about 35%, 40% or 45% to about 85%, or between about 50% to about 80%, e.g. between about 55%, 60% or 65% to about 70% or 75% of 4-HI based on the total weight of the extract, may be used.

Any one or combinations of these compounds and other similar compounds or fragments are hereinafter called “diabetes medicines” or “antidiabetics”.

The combination of the invention may be a combined preparation or a pharmaceutical or nutritional composition.

In one aspect of the invention, the combination of the invention, e.g. in the form of a nutritional composition, may comprise a soluble fiber, in particular pectin and/or beta-glucan. In particular there is provided a combination which comprises a cis-polyunsaturated fatty acid, at least one of phenylalanine, valine, arginine, leucine or isoleucine, pectin and beta-glucan.

The relative proportion of the active ingredients of the combination of the invention will, of course, vary considerably depending on the particular type of composition concerned, e.g. whether it is a liquid or solid form, or whether it is provided in pharmaceutical or nutritional form. All indicated proportions and relative weight ranges described herein are accordingly to be understood as being indicative of preferred or individually inventive teaching only and not limiting the invention in its broadest aspect.

The cis-polyunsaturated fatty acid(s) may be used in amount of about 10%, 15%, 30%, 35%, 40% or 45% to about 55% or 60%, e.g. about 10%, or about 15%, or about 50% by weight based on the total weight of fat present in the combination of the invention.

As cis-polyunsaturated fatty acid EPA: DHA may be used, e.g. in a ratio of about 0.1:10 to about 10:0.1, e.g. in a ratio of about 1:10 to about 10:1, preferably in a ratio of about 1.2 to about 0.8, in the combination of the invention. In a further aspect the combination of the invention may comprise about 20% or 25% to about 35% or 40%, e.g. about 30% EPA and about 10% or 15% to about 25% or 30%, e.g. about 20% DHA by weight based on the total weight of fat present in the combination of the invention.

In particular, EPA may be administered in an amount of about 200 mg, 300 mg, 400 mg or 500 mg to about 600 mg, 700 mg, 800 mg, 900 mg or 1000 mg, e.g. in an amount of about 300 mg per unit dose. DHA may be administered in an amount of about 100 mg, 200 mg, 300 mg, or 400 mg to about 500 mg, 600 mg, 700 mg, 800 mg, or 900 mg, e.g. in amount of about 200 mg per unit dose. One to five, e.g. two to three, unit doses may be administered per day.

The combination of the invention, e.g. in the form of a nutritional composition, may comprise about 5% en, 10% en, 15% en, 30% en, 40% en, 45% en or 50% en to about 55% en, 60% en, 65% en or 70% en, e.g. about 15% en or about 20% en or about 65% en as fat.

The combination of the invention, e.g. in the form of a nutritional composition, may comprise about 0.1% en to about 10% en, preferably about 0.5% en to about 5% en, more preferably about 1% en to about 2% en, even more preferably about 1.5% en to about 1.8% en as cis-PUFA.

In a further aspect the combination of the invention, e.g. in the form of a nutritional composition, may comprise about 10% en, 20% en, 25% en, or 30% en to about 35% en, 40% en, 50% en, 55% en or 60% en, e.g. about 32% en or about 35% en or about 55% en as amino-nitrogen source.

The combination of the invention, e.g. in the form of a nutritional composition, may comprise about 0.1% en to about 10% en, preferably about 0.5% en to about 5% en, more preferably about 1% en to about 2% en, even more preferably about 1.3% en to about 1.8% en, most preferably about 1.5% en as amino acid.

The ratio (weight/weight) of fat to protein in the combination of the invention, e.g. in the form of a nutritional composition, may be about 5:1 to about 1:10, or about 2:1 to about 1:2.

The amount of amino acids in free form or pharmaceutically acceptable salt form to be administered may be about 1%, 2%, 2.5%, 3%, 5%, 10%, 15% or 20% to about 25%, 30%, 35% or 40%, e.g. about 2% to about 30%, or about 20% to about 25% by weight based on the total weight of protein in the combination of the invention.

The ratio (weight/weight) of cis-PUFA to amino acid in the combination of the invention may be about 10:1 to about 1:10, or about 3:1 to about 1:5, or about 2:1 to about 1:3, or about 1:1 to about 1:1.6, or about 1:1, or about 1:2.

Corresponding to the needs of the single patient and under the proviso that it is intended by a physician to administer the combinations, e.g. the nutritional and pharmaceutical compositions, in separate compositions, it is possible to administer the antidiabetics as launched, e.g. nateglinide in the form as it is launched under the trademark Starlix™. If the drug metformin shall be administered in a separate pharmaceutical composition, it can be administered in the form as it is launched e.g. under the trademark DIABETOSAN™. If the drug metformin shall be administered in a separate pharmaceutical composition in the form of its hydrochloride salt, the metformin hydrochloride salt can be administered in the form as it is launched e.g. under the trademarks DIABETASE 500™, DIABETASE 850™ or GLUCOPHAGE S™.

In particular, a therapeutically effective amount of each of the combination partner of the combination of the invention, e.g. further comprising at least one diabetes medicine, may be administered simultaneously or sequentially and in any order, and the components may be administered separately or as a fixed combination. For example, the method according to the invention may comprise (i) administration of the cis-polyunsaturated fatty acid in free or oil or fat form and (ii) administration of the amino acid in free or pharmaceutically acceptable salt form or intact protein form, and optionally (iii) at least one diabetes medicine, e.g. nateglinide and/or a 4-HI source, simultaneously or sequentially in any order, in jointly therapeutically effective amounts, preferably in synergistically effective amounts, e.g. in daily or weekly dosages corresponding to the amounts described herein. The individual combination partners of the combinations as hereinabove described can be administered separately at different times during the course of therapy or concurrently in divided or single combination forms. The instant invention is therefore to be understood as embracing all such regimes of simultaneous or alternating treatment and the term “administering” is to be interpreted accordingly.

The effective dosage of each of the combination partners employed in the combinations as hereinabove described may vary depending on the particular compound or pharmaceutical or nutritional composition employed, the mode of administration, the condition being treated, the severity of the condition being treated. Thus, the dosage regimen for such combinations is selected in accordance with a variety of factors including the route of administration and the renal and hepatic function of the patient. A physician, clinician or veterinarian or dietician of ordinary skill can readily determine and prescribe the effective amount of the single active ingredients required to prevent, counter or arrest the progress of the condition.

According to the invention, the dosages of amino acids, e.g. arginine, may be comprised in the range of about 5 mg to about 150 mg/kg body weight/day, preferably from about 10 mg to about 100 mg/kg body weight/day, more preferably from about 20 mg to about 80 mg/kg body weight/day, more preferably from about 30 mg to about 60 mg/kg body weight/day.

According to the invention dosages of cis-polyinsaturated fatty acids may be in the range from about 1 mg to about 100 mg/kg body weight/day, preferably from about 5 mg to about 50 mg/kg body weight/day, more preferably from about 10 mg to about 40 mg/kg body weight/day, more preferably from about 15 mg to about 30 mg/kg body weight/day.

Dosages of the combination of amino acids plus cis-polyinsaturated fatty acids may be comprised in the range of about 10 mg to about 150 mg/kg body weight/day, preferably from about 20 mg to about 120 mg/kg body weight/day, more preferably from about 30 mg to about 100 mg/kg body weight/day.

The nature of diabetes and related diseases or conditions is multifactorial. Under certain circumstances, compounds with different mechanisms of action may be combined. However, just considering any combination of compounds having different mode of action but acting in the similar field does not necessarily lead to combinations with advantageous effects.

All the more surprising is the experimental finding that the combined administration of at least one cis-polyunsaturated fatty acid and at least one amino acid, and optionally at least one diabetes medicine, results not only in a beneficial, especially a synergistic, therapeutic effect but also in additional benefits resulting from combined treatment such as a surprising prolongation of efficacy, a broader variety of therapeutic treatment and surprising beneficial effects on diseases and conditions associated with diabetes, e.g. improved glucose clearance in response to insulin, enhanced triglyceride clearance, improved satiating effect, or less gain of weight.

It can be shown by established test models and especially those test models described herein that the combination of at least one cis-polyunsaturated fatty acid and at least one amino acid, and optionally at least one diabetes medicine, results in a more effective prevention or preferably treatment of diseases, especially metabolic disorders, and in particular type 2 diabetes mellitus and diseases and conditions associated with diabetes mellitus. In particular, it can be shown by established test models and especially those test models described herein that the combination of at least one cis-polyunsaturated fatty acid and at least one amino acid, results in a more effective prevention or preferably treatment of diseases, especially metabolic disorders, more especially diabetes and in particular type 2 diabetes mellitus, and diseases and conditions associated with diabetes.

In one aspect the present invention provides a method of treating metabolic disorders, more especially diabetes and in particular type 2 diabetes mellitus, or a disease or condition associated with diabetes as described hereinabove showing beneficial effects and additional benefits compared to monotherapy applying only one combination partner.

The person skilled in the pertinent art is fully enabled to select a relevant animal test model to prove the hereinbefore and hereinafter indicated therapeutic indications and beneficial effects. The pharmacological activity may, for example, be demonstrated following essentially an in-vivo test procedure in mice or in a clinical study as described in the Examples hereinafter.

Suitable clinical studies are in particular clinical double-blind, randomized, parallel-group studies in subjects with type 2 diabetes, e.g. inadequately controlled on diet or monotherapy alone.

These studies prove in particular the synergism of the claimed combinations, such as the combined preparations or pharmaceutical or nutritional compositions, respectively. The beneficial effects on diseases and conditions associated with diabetes mellitus as defined in this application can be determined directly through the results of these studies or by changes in the study design which are known as such to a person skilled in the art.

The combined administration of at least one cis-polyunsaturated fatty acid, at least one amino acid, optionally a soluble fiber and a non-glucose carbohydrate and optionally at least one diabetes medicine chosen from at least one of nateglinide, metformin or 4-HI results in a beneficial, especially a synergistic, therapeutic effect, especially on type 2 diabetes, and also in additional benefits such as a decrease of diabetes-related mortality, a surprising prolongation of efficacy of the drug, such as delaying the eventual need for insulin, a broader variety of therapeutic treatment, maintaining the target blood glucose level in type 2 diabetes patients, providing a good initial blood glucose control in type 2 diabetes patients, only modest changes in fasting plasma glucose level, and further surprising beneficial effects, comprising e.g. less or no gain of body weight, a decrease of gastrointestinal side effects or an improved safety profile, compared to a monotherapy applying e.g. only one of the pharmaceutically active compounds used in the combinations disclosed herein. In particular, the further surprising beneficial effects can also be observed during the treatment of metabolic disorders other than type 2 diabetes and during the treatment of diseases and conditions associated with type 2 diabetes. Further benefits are that lower doses of the individual drugs to be combined according to the present invention can be used to reduce the dosage, for example, that the dosages need not only often be smaller but are also applied less frequently, or can be used in order to diminish the incidence of side effects, such as anaemia, oedema or headache.

Furthermore, in a number of combinations as disclosed herein the side-effects observed with one of the components surprisingly do not accumulate on application of the combination.

The beneficial therapeutic effect, additional benefits and especially the surprising beneficial effects are observed in particular with a combination of at least one cis-polyunsaturated fatty acid and at least one amino acid and nateglinide and/or 4-HI. Very good results have been obtained with the combination of nateglinide and metformin or metformin hydrochloride, or the combination of nateglinide and a 4-HI source.

The beneficial therapeutic effects, additional benefits and also the surprising beneficial effects are observed especially in human subjects suffering from a more severe form of type 2 diabetes, i.e. human subjects having an elevated HbA_{1c} (glycosylated haemoglobin) value at baseline of greater 8% and more particular in human subjects having a HbA_{1c} value at baseline of greater than 9.5%, before treatment with the combinations described herein. If nateglinide is administered to such human patients, preferably, such human patients obtain a dose of between 90 and 200 mg, more preferably between 100 and 150 mg, for example 120 mg, nateglinide per meal as part of the combination given to them.

In one preferred embodiment of the invention, a dose of between 45 and 85 mg, more preferably 60 mg, of nateglinide per meal is administered as part of the combination to human subjects having a HbA_{1c} value at baseline between 6.8% and 8%, in particular between 6.8% and 7%. This provides the option to increase the amount of nateglinide later on, which option is advantegous especially in a situation when the HbA_{1c} value at baseline exceeds values of 7% after starting the treatment of the human subject for a period of time or constantly or if the responsible physician determines that the treatment schedule has to be changed to higher amounts of nateglinide for other reasons. One preferred combination partner in this embodiment is metformin or a 4-HI source.

A pharmaceutical composition for combination therapy comprising nateglinide and metformin in a pharmaceutical carrier, which is preferably in the form of a tablet, a capsule, a suspension or a liquid, contains most preferably from about 100 mg to about 130 mg of nateglinide and from about 320 mg to about 1500 mg, more preferably 330 mg to 350 mg, metformin per dose unit.

A pharmaceutical or nutritional composition for combination therapy comprising a 4-HI source, e.g. substantially pure 4-HI, may contain from about 10 to about 100 mg per kg body weight, e.g. about 500 mg to about 1 g per daily dose.

Furthermore, the beneficial therapeutic effects, additional benefits and also the surprising beneficial effects are observed especially in human subjects having a body mass index (BMI) of 20 to 35 kg/m², in particular a BMI of 27 to 35 kg/m², and even more enhanced in human subjects with a BMI of 30 to 35 kg/m². Human subjects having a BMI greater 30 kg/m² are defined to be clinically obese.

Additionally, the beneficial therapeutic effects, additional benefits and also the surprising beneficial effects are observed especially in patients poorly controlled by monotherapy with one of the components of the combinations disclosed herein.

In one aspect of the invention there is provided a combination of the invention in form of a pharmaceutical or nutritional composition. Preferably, nutritional compositions may be used. Compositions in accordance with the present invention may be employed for administration in any appropriate manner, e.g. enterally, e.g. orally, for example in liquid form or in solid form, preferably in liquid form. Optionally the compositions may be administered in the form of a tube feeding solution.

Pharmaceutical compositions for oral administration are, for example, those in single dose unit forms, such as dragées, tablets, e.g. coated tablets, capsules, e.g. soft gel, or sachets. Pharmaceutical compositions may further be provided in the form of syrups, liquid suspensions, emulsions and solutions in convenient dosage forms. They are prepared in a manner known per se, for example by means of conventional mixing, granulating, sugar-coating, confectioning, dissolving or lyophilizing processes. It will be appreciated that the unit content of a combination partner contained in an individual dose of each dosage form need not in itself constitute an effective amount since the necessary effective amount can be reached by administration of a plurality of dosage units.

Suitable physiologically acceptable carriers for preparation of oral dosage forms may be especially fillers, such as sugars, for example lactose, mannitol or sorbitol, cellulose preparations and/or calcium phosphates, for example tricalcium phosphate or calcium hydrogen phosphate, and also binders, such as starch pastes using, for example, corn, wheat, rice or potato starch, gelatin, tragacanth, methylcellulose and/or polyvinylpyrrolidone, and, if desired, disintegrators, such as the above-mentioned starches, and also carboxymethyl starch, cross-linked polyvinylpyrrolidone, agar, or alginic acid or a salt thereof, such as sodium alginate. Further excipients may be especially flow-conditioners and lubricants, for example silicic acid, talc, stearic acid or salts thereof, such as magnesium or calcium stearate, and/or polyethylene glycol. Dragée cores are provided with suitable coatings, there being used inter alia concentrated sugar solutions which may contain gum arabic, talc, polyvinylpyrrolidone, polyethylene glycol and/or titanium dioxide, or coating solutions in suitable organic solvents or solvent mixtures. Dyes or pigments may be added to the tablets or dragée coatings, for example for identification purposes or to indicate different doses of active ingredient.

Other orally administrable compositions may be in the form of hard gelatin capsules or soft, sealed capsules consisting of gelatin and a plasticizer, such as glycerol or sorbitol. The hard gelatin capsules may comprise the composition of the invention in the form of granules, for example in admixture with fillers, such as lactose, binders, such as starches, and/or glidants, such as talc or magnesium stearate, and, if desired, stabilizers. In soft capsules the composition of the invention is preferably dissolved or suspended in suitable liquids, such as fatty oils, paraffin oil or liquid polyethylene glycols, it is likewise being possible to add stabilizers.

The pharmaceutical compositions of the invention may consist exclusively of at least one cis-polyunsaturated fatty acid and at least one amino acid, and optionally at least one diabetes medicine. They may further comprise at least one pharmaceutically acceptable carrier.

Alternatively, the combination of the invention may be provided in form of a nutritional composition, e.g. in form of a dietary supplement, or a medical food, e.g. in form of a complete meal, a part of a meal, or a food additive, or beverage, e.g. in form of a powder for dissolution. The powder may be combined with a liquid, e.g. water, or other liquid, such as milk or fruit juice, to obtain a ready-to-consume composition, e.g. ready-to-drink composition or instant drink. Alternatively, the beverage may be a soft drink, juice, milk-shake, yogurt drink, smoothie or soy-based drink. The nutritional compositions may be in form of a bar, or dispersed in foods of any sort, such as baked products, cereal bars, dairy bars, snack-foods, soups, breakfast cereals, müesli, candies, tabs, cookies, biscuits, crackers, such as a rice crackers, and dairy products.

The nutritional compositions of the invention in form of dietary means, e.g. supplements, may consist exclusively of at least one cis-polyunsaturated fatty acid and at least one amino acid, and optionally at least one diabetes medicine. They may further comprise at least one nutritionally acceptable carrier.

Suitable product formats according to the present invention include solution, ready-for-consumption composition, e.g. ready-to-drink compositions, instant drink, liquid comestibles, like soft drinks, juice, sports drinks, milk drinks, milk-shakes, yogurt drinks or soup. In a further embodiment of the invention, the compositions of the present invention may be manufactured and sold in the form of a concentrate, a powder, or granules, e.g. effervescent granules, which are diluted with water or other liquid, such as milk or fruit juice, to yield a ready-for-consumption composition, e.g. ready-to-drink compositions or instant drink.

Optionally, the compositions according to the invention may be nutritionally complete, i.e. may include vitamins, minerals, trace elements as well as additional nitrogen, carbohydrate and additional fatty acid sources so that they may be used as the sole source of nutrition supplying essentially all the required daily amounts of vitamins, minerals, carbohydrates, fatty acids, proteins and the like. Accordingly, the compositions of the invention may be provided in the form of a nutritionally balanced complete meal, e.g. suited for oral or tube feeding. Preferably the compositions of the invention are for oral administration.

The nutritional recommendations for individuals with type 1 and type 2 diabetes are about 10 to about 20 en % protein, less than about 10 en % saturated fatty acids, about 5 to about 10 en % polyunsaturated fatty acids, and the remaining calories to be divided between carbohydrate, e.g. about 40 to about 50 en % carbohydrate, and monounsaturated fatty acids, e.g. about 10 to abut 30 en % monounsaturated fatty acids, based on individual medical needs and personal tolerance.

According to the present invention nutritional compositions comprising about 5% en, 10% en, 15% en, 30% en, 40% en, 45% en or 50% en to about 55% en, 60% en, 65% en or 70% en, e.g. about 15% en or about 20% about or about 65% en as fat and about 10% en, 20% en, 25% en, or 30% en to about 35% en, 40% en, 50% en, 55% en, 60% en, e.g. about 32% en or about 35% en or about 55% en, as amino-nitrogen source may be used.

As carbohydrates preferably soluble fibers, as defined hereinabove, preferably guar gum, e.g. partially hydrolyzed guar gum, may be used in combination with a non-glucose carbohydrate, as defined hereinabove, preferably galactose and/or fructose. The non-glucose carbohydrate, preferably galactose and/or fructose, and soluble fiber, preferably guar gum, may be used in a ratio of about 100 to about 0.1, e.g. in a ratio of about 100 to about 1.

In one aspect of the invention the combinations of the invention may comprise a mixture of soluble fibers comprising e.g. beta-glucan and pectin, e.g. in an amount of about 0.1 to about 10% by weight based on the total weight of the composition. Pectin and beta-glucan may be used in a ratio of about 20 to about 0.05, suitably about 10 to about 0.1, more suitably about 5 to about 0.5, e.g. about 2 to about 1.

In another aspect of the invention the nutritional compositions of the invention comprise low, e.g. between about 2.5% en or about 5% en to about 7.5% en or 10% en, e.g. about 5% en as carbohydrate.

In one aspect the present invention also pertains to a combination comprising at least one cis-polyunsaturated fatty acid, at least one amino acid, a soluble fiber, e.g. guar gum, and a non-glucose carbohydrate, e.g. galactose.

It may be desirable to provide the nutritional compositions of the invention in the form of a low calorie meal replacement or other nutritional product. Suitably, a single serving of a low calorie meal replacement will have a caloric value of less than about 1000 kcal (4.2 MJ), and preferably between about 200 kcal (0.8 MJ) and about 500 kcal (2.1 MJ). Suitable low calorie nutritional product may include any nutritional product described hereinabove.

Conventional additives may be included in the pharmaceutical or nutritional compositions of the invention, including any of those selected from preservatives, chelating agents, osmotic agents, buffers or agents for pH adjustment, effervescing agents, sweeteners, e.g. artificial sweeteners, flavoring agents, coloring agents, taste masking agents, acidulants, emulsifiers, stabilizers, thickening agents, suspending agents, dispersing or wetting agents, antioxidants, acidulants, texturizers, antifoam agents, and the like. For example the pharmaceutical or nutritional compositions of the invention may contain curcumin, chlorogenic acid or cinnamon.

According to the invention, the pharmaceutical or nutritional compositions of the invention may comprise natural botanical materials, such as Phaseolamin (bean) Lupin extract, vanadium and/or Fenugreek.

In addition to the foregoing the present invention also provides a process for the production of a composition, e.g. nutritional or pharmaceutical formulation, as hereinbefore defined, which process comprises bringing the individual components thereof into intimate admixture and, when required compounding the obtained composition in a food or beverage product, for example ready-made drink, or in unit dosage form, for example filling said composition into a sachet.

Dependent on the form of application of the pharmaceutical or nutritional compositions of the invention, i.e. as complete meal, part of a meal, food additive, drink, sachet, tablet or capsule, the compositions of the invention may be taken once daily to e.g. five times daily. Preferably the unit doses are taken five or three times, e.g. with the main meals, e.g. without restriction to time of day. Preferably, the unit doses are taken together with, or shortly before, e.g. 15 minutes before, the main meals, e.g. in the morning, at noon, and in the evening.

The combination, e.g. combined preparation, e.g. pharmaceutical or nutritional compositions, of the invention comprising at least one cis-polyunsaturated fatty acids and at least one amino acid may be self-administered.

The combination, e.g. combined preparation, e.g. pharmaceutical or nutritional compositions, of the invention comprising at least one cis-polyunsaturated fatty acids and at least one amino acid and at least one diabetes medicine may be administered under the supervision of a medical specialist.

For prevention, delay of progression or treatment of metabolic disorders and in particular type 2 diabetes and disease and conditions associated with diabetes under clinical supervision it is possible to further combine the combinations as described hereinabove with weight-control drugs. For example, the combinations may be provided in the form of a kit for separate, sequential or simultaneous administration in conjunction with weight-control drugs such as amphetamines, fenfluramine, phenylpropanolamine, or mazindol. The weight-control drugs may conveniently be formulated together with the combinations as hereinabove described, e.g. combinations comprising at least one cis-polyunsaturated fatty acids and at least one amino acid and optionally at least one diabetes medicine, in one combined unit dosage form, which may also be a fixed combination.

In one aspect of the present invention the combination of the invention comprising at least one cis-polyunsaturated fatty acids and at least one amino acid, optionally further comprising a soluble fiber, e.g. guar gum, and a non-glucose carbohydrate, e.g. galactose, may be co-administered together with nateglinide and/or metformin. In another aspect of the present invention the combination of the invention comprising at least one cis-polyunsaturated fatty acids and at least one amino acid, optionally further comprising a soluble fiber, e.g. guar gum, and a non-glucose carbohydrate, e.g. galactose, may be co-administered together with nateglinide and/or 4-HI.

In a further aspect, the present invention provides a commercial package comprising as active ingredients a combination of the invention comprising at least one cis-polyunsaturated fatty acids and at least one amino acid, and optionally at least one diabetes medicine chosen from at least one of nateglinide, metformin or a 4-HI source, together with instructions for simultaneous, separate or sequential use thereof in the prevention, delay of progression or treatment of metabolic disorders and in particular type 2 diabetes and disease and conditions associated with diabetes.

Optimally, the combination of the invention comprising at least one cis-polyunsaturated fatty acids and at least one amino acid, optionally further comprising a diabetes medicine, is consumed at least once a day on a regular basis until for example normal weight or a blood glucose level of 180 mg/dL or less 2 hours after meals has been resumed. When the supplement is supplied in the form of a food or beverage, a suitable serving size may be in the range 20 to 500 g, preferably 50 to 250 g. If provided in the form of a meal or in pharmaceutical form, one or several dosages of the combination of the invention comprising at least one cis-polyunsaturated fatty acids and at least one amino acid, optionally further comprising a diabetes medicine, may be administered over a 24-hour period. Since these formulations are safe to consume, obese or overweight subjects or subjects with diabetes, can continue taking these supplements for as long as required, and preferably until normal weight or a blood glucose level of 180 mg/dL or less 2 hours after meals has been resumed.

Anyone perceived to be at risk from metabolic disorders and in particular type 2 diabetes and disease and conditions associated with diabetes or already suffering from these or associated disorders, can benefit from ingesting the combinations, e.g. compositions, of the invention. By triggering insulin secretion and hence glucose and/or triglyceride clearance, and/or promoting satiety, the compositions of the invention have also the effect of counteracting the sequelae of long-term complications of diseases and conditions associated with diabetes mellitus.

According to the invention it is possible to effectively ameliorate symptoms and conditions associated with metabolic disorders and in particular type 2 diabetes and disease and conditions associated with diabetes with compounds, which do not show any severe side effects. For example, the present methods are well-tolerated and simple to apply. The present methods improve the quality of life.

The utility of all the combinations, e.g. compositions, of the present invention may be observed in standard clinical tests in, for example, known indications, for example using nutritional compositions as described in the Examples hereinbelow, for example using cis-PUFA in the range as hereinabove described, e.g. of 1% en to 70% en or 40% en to 70% en, e.g. 1.5% en or 50% en, and protein in the range as hereinabove described, e.g. of 1% en to 60% en, e.g. 1.5% en or 30% en in a nutritional composition, e.g. for co-administration with a pharmaceutical composition comprising nateglinide dosages in the range of 100 mg to 130 mg per unit dose, and/or metformin dosages in the rage of 320 mg to 1500 mg per unit dose, and/or 4-HI dosages in the range of 10 mg to 100 mg per kg body weight for a mammal, e.g. adult and in standard animal models. The increased insulin secretion/glucose and/or triglyceride clearance/promotion of satiety provided by the compositions may be observed in standard animal tests and in clinical trials, e.g. as described in the Examples below.

One human clinical trial may be affected as follows:

A single blind, placebo controlled, parallel study in 90 subjects may be performed using the combination of the invention, e.g. comprising cis-PUFA in the range of as hereinabove described, e.g. of 1% en to 70% en or 40% en to 70% en, e.g. 1.5% en or 50% en, and protein in the range as hereinabove described, e.g. of 1% en to 60% en, e.g. 1.5% en or 30% en, e.g. co-administered with a pharmaceutical composition comprising nateglinide dosages in the range of 100 mg to 130 mg per unit dose, and/or metformin dosages in the rage of 320 mg to 1500 mg per unit dose, and/or 4-HI dosages in the range of 10 mg to 100 mg per kg body weight, to study the effects on weight loss, weight maintenance after weight loss and metabolic syndrome characteristics. The following parameters may be assessed at baseline and after 3 month of treatment: body weight, weight regain and composition of weight regain, attitude towards eating, appetite profile, OGT, glucose, insulin, C-peptide, TG, Glycerol, FFA and satiety (all subjects).

Another human clinical trial may be affected as follows:

To evaluate the efficacy of glucose control and insulin response of combinations of the invention vs control, combinations of the invention, e.g. comprising cis-PUFA in the range of about 1% en to 70% en, e.g. 40% en to 70% en, e.g. 1.5% en, 2% en, 5% en, 10% en, 20% en, 40% en, 50% en, 60% en or 65% en and protein in the range of 1% en, 10% en or 20% en to 40% en or 60% en, e.g. about 1.5% en or about 30% en, e.g. co-administered with a pharmaceutical composition comprising nateglinide dosages in the range of 100 mg to 130 mg per unit dose, and/or metformin dosages in the rage of 320 mg to 1500 mg per unit dose, and/or 4-HI dosages in the range of 10 mg to 100 mg per kg body weight, are administered for 3 weeks to patients with type 2 diabetes. Glycemic and insulin response to a carbohydrate load of 75 g at day 1 and after treatment at day 21 are measured.

The invention will now be further illustrated by the following Examples.

### EXAMPLE 1

The effects of specific polyunsaturated fatty acids (PUFA), both n-6 vs n-3 PUFA, on insulin sensitivity and lipid risk factors influencing insulin resistance, in a Type II diabetes animal model namely, Lepr db/db mice were investigated.

Methods—Animals, Diets and Design:

C57BLKS/J-Lepr^{db/db} male mice (4 wks of age) were obtained from Jackson Laboratory, Bar Harbor, Me. Mice were initially maintained on commercial chow for 2 wks, followed by a purified stabilization diet providing 40% en as fat and 2% en as linoleic acid (LA, 18:2n-6) for another 2 wks. At 8 wks of age, a baseline oral glucose tolerance test was conducted. Based on their OGTT profile, mice were evenly distributed among 6 groups (n=6 per each group) and were fed one of 6 experimental diets designed to establish the relative importance of specific fatty acid supplementation on ameliorating insulin resistance.

Table 1 gives the predicted fatty acid profile (% en) for the 6 experimental diets. The control diet, based on butter and palm oil blend, provided 40% en as fat with 2% en as LA. In addition to the saturated fat control” with its basal level of 2% en as 18:2, each of the 5 test diets provided an additional 2% en as a specific PUFA, namely LA (LA group), linolenic acid (LNA group), docosahexaenoic acid (DHA group), eicosapentaenoic acid+docosahexaenoic acid (EPA+DHA group, 1.2% en as EPA and 0.8% as DHA) and DHA+arachidonic acid (DHA+AA group, 1% en as DHA and 1% en as AA). These Lepr db/db mice were fed one of the above 6 diets for 6 weeks.

TABLE 1
Expected fatty acid content (% en) of experimental diets

| Fatty acids | | | | | EPA + | DHA + |
|---|---|---|---|---|---|---|
| (% en in diet) | Control | LA | LNA | DHA | DHA | AA |
| 12:0 | 0.7 | 0.7 | 0.7 | 0.7 | 0.5 | 0.7 |
| 14:0 | 2.6 | 2.8 | 2.8 | 2.5 | 2.3 | 2.5 |
| 16:0 | 13.7 | 12.1 | 12.0 | 13.2 | 14.1 | 13.5 |
| 18:0 | 3.5 | 3.6 | 3.7 | 2.9 | 3.2 | 3.2 |
| SFA | 20.5 | 19.3 | 19.2 | 19.2 | 20.1 | 19.9 |
| 18:1 | 13.4 | 12.3 | 12.5 | 13.3 | 13.5 | 13.4 |
| MUFA | 13.4 | 12.3 | 12.5 | 13.3 | 13.5 | 13.4 |
| 18:2n-6 | 2.1 | 2.2 | 2.2 | 2.1 | 2.2 | 2.1 |
| 18:3n-3 | 0.2 | 2.0 | 2.0 | 0.1 | 0.2 | 0.2 |
| 20:4n-6 | Trace | Trace | Trace | Trace | Trace | Trace |
| 20:5n-3 | Trace | Trace | Trace | Trace | 1.2 | Trace |
| 22:6n-3 | Trace | Trace | Trace | 2.1 | 0.8 | 1.0 |
| PUFA | 2.3 | 4.3 | 4.3 | 4.3 | 4.4 | 3.3 |
| SFA:PUFA | 9.0 | 4.5 | 4.5 | 4.4 | 4.6 | 6.0 |

Body weights of animals were recorded each week. Insulin tolerance testing was performed after 6 weeks of dietary intervention. Mice (non-fasted) were injected intraperitoneally with 1.5 U/kg human insulin and blood glucose concentrations were determined at 0, 15, 30 and 60 min after insulin injection by tail bleeding, using a glucometer. A week later, fasting blood samples were collected by cardiac puncture, under Co₂/O₂ anesthesia, for analysis of plasma insulin, triglyceride (TG) and total cholesterol (TC). Plasma TC and TG were determined by enzymatic assay using Sigma kits #362 and #336, respectively (Sigma Diagnostics Co, St. Louis, Mo.). Plasma insulin was determined using the RIA kit (Linco research Inc, MO).

Results

Table 2 shows the body weights of mice at the beginning of the study and 6 wks after dietary intervention along with weight gain during intervention. Weight gain was not significantly different among groups.

TABLE 2
Body weight and weight gain Lepr db/db mice fed different fatty acids

| | Control | LA | LNA | DHA | EPA + DHA | DHA + AA |
|---|---|---|---|---|---|---|
| | (n = 5) | (n = 6) | (n = 4) | (n = 6) | (n = 6) | (n = 6) |
| Initial body wt (g) | 43.0 ± 2.6 | 41.5 ± 2.1 | 42.2 ± 3.1 | 42.8 ± 2.5 | 42.9 ± 2.8 | 42.8 ± 2.7 |
| Body wt at 6 wks | 45.7 ± 1.4 | 43.9 ± 5.4 | 50.5 ± 2.4 | 48.9 ± 3.8 | 50.6 ± 4.1 | 47.5 ± 3.3 |
| (g) | | ^{a} | ^{b} | | | |
| Wt gain at 6 wks | 2.7 ± 3.6 | 2.5 ± 4.3 | 8.3 ± 4 | 6.1 ± 5.2 | 7.7 ± 4.2 | 4.7 ± 6.2 |
| (g) | | | | | | |

^{a,b} Means in a row sharing different superscript letters are significantly different (p < 0.05) using one-way ANOVA and Fisher's PLSD test.

Table 3 shows insulin tolerance data after 6 wk for Lepr db/db mice fed these different diets EPA+DHA improved insulin sensitivity in Lepr db/db mice as evidenced by enhanced blood glucose clearance following insulin administration. EPA+DHA reduced blood glucose concentration by 10% of initial values after 30 min of insulin injection. The decrease was significantly lower than the controls with no PUFA supplement. By 60 min, blood glucose fell below initial values for all PUFA diets. EPA+DHA resulted in 26% decline.

TABLE 3
Mean blood glucose values (percentage of intial) after insulin
administration in Lepr db/db mice

| | 0 | 15 min | 30 min | 60 min |
|---|---|---|---|---|
| Control | 100 | 179 | 148 | 116 |
| LA | 100 | 135 | 115 | 85 |
| LNA | 100 | 138 | 127 | 100 |
| DHA | 100 | 131 | 120 | 99 |
| EPA + DHA (Fish | 100 | 113 | 90 | 74 |
| oil) | | | | |
| DHA + AA | 100 | 129 | 115 | 95 |

Table 4 shows fasting plasma TC and TG of Lepr db/db mice. Again, EPA+DHA resulted in lowest fasting plasma TG compared to all other PUFA. Fasting plasma TC was not different among diet groups.

TABLE 4
Fasting plasma lipids in Lepr db/db mice fed different fatty acids

| | Control | LA | LNA | DHA | EPA +DHA | DHA +AA |
|---|---|---|---|---|---|---|
| TG | 106 ± 33^{a,c} | 120 ± 24^{a,b} | 142 ± 27^{b} | 101 ± 14^{a,c} | 79 ± 8^{c} | 101 ± 21^{a,c} |
| (mg/dl) | | | | | | |
| TC | 284 ± 25 | 313 ± 28 | 326 ± 13 | 288 ± 39 | 294 ± 21 | 289 ± 37 |
| (mg/dl) | | | | | | |

^{a,b,c}Means in a row sharing different superscript letters are significantly different (p < 0.05) using one-way ANOVA and Fisher's PLSD test.

Discussion

EPA+DHA improved glucose clearance in response to insulin in the Type II diabetes mouse model Lepr db/db. Enhanced glucose disposal indicates that EPA+DHA improved insulin sensitivity, even in extremely overt Type II diabetes mellitus in this model. Furthermore, low plasma TG in EPA+DHA-fed mice provides evidence for enhanced TG clearance. In summary, the DHA+EPA combination revealed a substantial benefit on insulin resistance.

### EXAMPLE 2

Acute effects of a nutritional formula, Starlix® and 4-HI on glycemic control in the type 2 diabetes animal model (Lepr db mouse) were studied. Animals underwent an OGT (1 g glucose/kg) combined with oral administration of the active ingredient. The following groups were tested: (A) placebo (2% Tween 80, 20 ml/kg), (B) 4-HI (100 mg/kg), (C) guar gum (150 mg/kg), (D) Starlix® (65 mg/kg), (E) Starlix®+4-HI, and (F) Starlix®+guar gum. Postprandial glucose and insulin were studied at 0, 30, 60 and 90 min.

Results:

Table 5—Effect on serum glucose levels. Starlix® showed a significant hypoglycemic effect at T90. In combination with 4-HI, significant hypoglycemic effects occurred at all time points.

TABLE 5

| | 0 | 30 | 60 | 90 |
|---|---|---|---|---|
| Placebo | 212.7 | 463.7 | 564.7 | 727.7 |
| 4HI | 227.7 | 473.7 | 494.7 | 584 |
| Guar | 212.7 | 487.3 | 615.7 | 711.7 |
| Starlix | 210 | 456.7 | 497.3 | 599 |
| 4HI & Starlix | 208 | 379.7 | 398 | 448.7 |
| Guar & Starlix | 205.3 | 499 | 588.3 | 660.3 |

TABLE 6
Effect on serum insulin levels. Starlix, guar gum, and a combination of
both were active in lowering serum insulin.

| | 0 | 30 | 60 | 90 |
|---|---|---|---|---|
| Placebo | 8.38 | 11.91 | 6.39 | 4.82 |
| 4HI | 8.17 | 11.27 | 6.41 | 4.63 |
| Guar | 8.38 | 9.66 | 6 | 3.69 |
| Starlix | 9.8 | 11.18 | 5.38 | 3.75 |
| 4HI & Starlix | 8.49 | 11.76 | 5.8 | 4.13 |
| Guar & Starlix | 8.63 | 9.67 | 7.63 | 4.99 |

TABLE 7
Summary of significant changes vs placebo

| | Glucose | Insulin |
|---|---|---|
| 4-HI | | |
| Guar gum | | ↓T30 |
| Starlix | ↓T90 | ↓T60 |
| 4-HI + Starlix | ↓T30, T60, T90 | |
| Starlix + Guar | | ↓T30 |

↓= decrease.
Starlix and Starlix + 4-HI showed a significant hypoglycemic effect indicating improved insulin sensitivity.

### EXAMPLE 3

Material and Methods

Animals, diets and basic design. Male Syrian hamsters (initial body weight 120-130 g) obtained from Charles River Breeding Labs (Wilmington, Mass.) were use in the study. The animals were randomly assigned to one of four group (n=7-9) and fed purified Atkins or Ornish diets containing either an American Fat Blend (AFB) or Smart Balance fat (see Tables 7 and 8). After an initial 8 wk feeding of diets with these two different fats (with no fat effect), both diets were continued for an additional 4 wk comparison while all feeding them the same (AFB) fat within their assigned diet type. The animals were housed in 2-3 per cage and kept in a temperature-controlled environment with a 12-hour light/dark cycle. All hamsters had free access to water, and fresh diet was provided daily. Body weight was monitored on a weekly basis, and food intake on a daily basis. The Brandeis University Animal Care and Use Committee approved all protocols and procedures. At the end of each diet period, hamsters were fasted overnight (15 h) and blood was collected via cardiac puncture under CO₂/O₂ anesthesia for plasma lipid and lipoprotein analysis.

Plasma lipid analysis. Total plasma cholesterol and triglycerides were determined by enzymatic assays (Sigma Diagnostics kit, procedure #352 for cholesterol and #336 for triglycerides, Sigma Chemicals, St Louis, and MO

TABLE 8
Atkins vs Ornish, no cholesterol, American Fat Blend
vs Smart Balance fat. Diet g/kg.

| | Ornish | Ornish | Atkins | Atkins |
|---|---|---|---|---|
| Ingredient | AFB | Smt Bal | AFB | Smt Bal |
| Casein | 180 | 150 | 425 | 425 |
| Dextrose | 297 | 295 | — | — |
| Cornstarch | 270 + 50 | 270 + 50 | 50 | 50 |
| Cellulose | 100 | 100 | 100 | 100 |
| Fat: | | | | |
| Tallow | 30 | — | 210 | — |
| Butter | 12 | — | 88 | — |
| Lard | 10 | — | 70 | — |
| Smt Bal (69% | — | 72.5 | — | 507 |
| fat) | | | | |
| Mineral Mix | 41 | 41 | 58 | 58 |
| Vitamin Mix | 10 | 10 | 14 | 14 |
| Choline chloride | 2.1 | 2.1 | 3 | 3 |

50 g cornstarch and 800 ml of water for gel per kg diet

TABLE 9
Atkins vs Ornish, no cholesterol, AFB. Diet g/kg.

| Ingredient | Ornish | Atkins |
|---|---|---|
| Casein | 180 | 425 |
| Dextrose | 297 | — |
| Cornstarch | 270 + 50 | 50 |
| Cellulose | 100 | 100 |
| Fat: | | |
| Tallow | 30 | 210 |
| Butter | 12 | 88 |
| Lard | 10 | 70 |
| Smt Bal (69% fat) | | |
| Mineral Mix | 41 | 58 |
| Vitamin Mix | 10 | 14 |
| Choline chloride | 2.1 | 3 |

50 g cornstarch plus 800 ml water per kg for gel

Results

The fat and protein-rich Atkins diet produced lower body weight than the low-fat, carbohydrate-rich Ornish diet, the difference being significant at week 12 (Table 9). Plasma cholesterol (TC) for hamsters fed the Atkins diet was always lower the Ornish diet, being significant at week 5 (Table 9).

TABLE 10
Body weight and plasma lipids of hamsters fed Atkins or Ornish
diets for 5-12 weeks.

| Body weight | Atkins | Ornishs |
|---|---|---|
| Initial (g) | 125 ± 9 | 129 ± 10 |
| at week 5 (g) | 153 ± 17 | 162 ± 15 |
| at week 12 (g) | 157 ± 18^{a} | 171 ± 18^{a} |
| intake, g/day (kcal/day) | 9.8 ± 0.6 (49.5 ± 3.0) | 13.2 ± 1.2 (47.8 ± 4.3) |

### EXAMPLE 4

Mixture of fat, protein and carbohydrate for a nutritional supplement effective against metabolic disorders.

| | Percent Energy |
|---|---|
| Canola Oil | 10 |
| MCT Oil | 4 |
| EPAX ® 4510 (Marine Oil) | 30 |
| DHA Gold ® (Algae Oil) | 20 |
| Ca Caseinate | 20 |
| Arginine | 3.1 |
| Leucine | 2.5 |
| Valine | 1.9 |
| Phenylalanine | 2.0 |
| Isoleucine | 2.5 |
| Corn Syrup (25DE) | 4 |
| Total | 100 |

### EXAMPLE 5

Mixture of Example 4 for co-administration with a pharmaceutical composition comprising nateglinide (120 mg)

| | |
|---|---|
| nateglinide | 120 mg |
| lactose monohydrate | 283 mg |
| microcrystalline cellulose | 142 mg |
| Povidone | 24 mg |
| croscarmellose sodium | 36.8 mg |
| magnesium stearate | 11.4 mg |
| opadry yellow | 18.0 mg |
| colloidal silicon dioxide | 12.8 mg |

### EXAMPLE 5

Nutritional Formulation, Containing Per Serving:

| | | |
|---|---|---|
| casein/whey | 21.4 | g |
| arginine | 0.6 | g |
| leucine | 1.51 | g |
| phenylalanine | 0.81 | g |
| resistant starch | 7.0 | g |
| fructose | 4.0 | g |
| Hydrolysed guar gum ⁽¹⁾ | 3.0 | g |
| Canola Oil | 3.0 | g |
| Vitamin mix ⁽²⁾ | | |
| Mineral mix ⁽³⁾ | | |
| Carnitine | | |
| Taurine | | |
| Inositol | | |
| Lipoic Acid | | |
| Betain/choline | | |
| Fenugreek extract | | |

Energy per serving: 160K cal.
⁽¹⁾ Benefiber ®, from Novartis Nutrition Corporation
⁽²⁾ comprises Vitamin C, Vitamin E, Vitamin B12, Vitamin B, Vitamin B1, Vitamin B2, Folic Acid.
⁽³⁾ comprises Chromium, Magnesium and Potassium.

1. A combination comprising
(a) at least one of linolenic, linoleic, conjugated linoleic acid, arachidonic, eicosapentaenoic acid or docosahexaenoic acid,
(b) at least one of phenylalanine, valine, arginine, leucine or isoleucine in free and/or salt form, and optionally
(c) at least one diabetes medicine chosen from at least one of nateglinide, metformin or a 4-hydroxy-isoleucine source.

2. A combination of claim 1 wherein (a) comprises eicosapentaenoic acid and docosahexaenoic acid.

3. A combination of claim 1 further comprising a soluble fiber and/or non-glucose carbohydrate.

4. A combination of claim 3 wherein the soluble fiber is guar gum and the non-glucose carbohydrate is galactose.

5. A combination of claim 1 further comprising pectin and beta-glucan.

6. A combination of claim 1 wherein (c) is nateglinide.

7. A combination of claim 1 wherein (c) is a combination of nateglinide and a 4-HI source.

8. A method of improving the bodily appearance of a mammal which comprises orally administering to said mammal a combination according to claim 1 in a dosing effective to influence the glucose metabolism, and repeating said dosing until a cosmetically beneficial loss of body weight has occurred.

9. Use of a combination according to claim 1 for the preparation of a medicament for the prevention, delay of progression or treatment of metabolic disorders, more especially diabetes or a disease or condition associated with diabetes.

10. A pharmaceutical or nutritional composition comprising a quantity, which is jointly therapeutically effective against metabolic disorders, of a combination according to claim 1, and at least one pharmaceutically or nutritionally acceptable carrier.

11. Use of a combination according to claim 1 for the cosmetic treatment of a mammal in order to effect a cosmetically beneficial loss of body weight.

12. A method of improving the bodily appearance of a mammal which comprises orally administering to said mammal a composition according to claim 10.

13. Use of a composition according to claim 10 for the preparation of a medicament for the prevention, delay of progression or treatment of metabolic disorders, more especially diabetes or a disease or condition associated with diabetes.

14. A commercial package comprising as active agents at least one cis-polyunsaturated fatty acid, at least one amino acid, and optionally at least one diabetes medicine selected from the group consisting of nateglinide, metformin, a 4-HI source, together with instructions for simultaneous, separate or sequential use thereof in the prevention, delay of progression or treatment of metabolic disorders or in a method of improving the bodily appearance of a mammal.

15. A fixed combination comprising
(a) a cis-polyunsaturated fatty acid chosen from at least one of linolenic, linoleic, arachidonic, eicosapentaenoic acid or docosahexaenoic acid, and
(b) an amino acid chosen from at least one of phenylalanine, valine, arginine, leucine or isoleucine for co-administration with

16. A nutritional composition according to claim 10 comprising about 40% en to about 70% en, as fat and about 20% en to about 40% en as amino-nitrogen source, optionally in combination with a diabetes medicine.

17. A nutritional composition according to claim 10 comprising about 15% en to about 70% en, as fat and about 20% en to about 60% en as amino-nitrogen source, optionally in combination with a diabetes medicine.
